(19) United States
(12) Patent Application Publication
(10) Pub. No.: US 2010/0216184 A1
(43) Pub. Date: Aug. 26, 2010

(54) PREPARATION OF CELL EXTRACT AND ITS APPLICATION FOR CELL-FREE PROTEIN SYSTHESIS

(76) Inventors: Dong-Myung Kim, Daejeon (KR); Tae-Wan Kim, Seoul (KR); Chang-Gil Park, Daejeon (KR); Cha-Yong Choi, Seoul (KR)

Correspondence Address: IPLA P.A., 3550 WILSHIRE BLVD., 17TH FLOOR, LOS ANGELES, CA, 90010, US

(21) Appl. No.: 12/279,835

(22) PCT Filed: Feb. 15, 2007
(86) PCT No.: PCT/KR2007/000799

§ 371 Date: Aug. 18, 2008

(30) Foreign Application Priority Data
Feb. 17, 2006 (KR) 10-2006-0015605

(51) Int. Cl. C12P 21/06

(52) U.S. Cl. 435/68.1

(57) ABSTRACT

Disclosed is a process for simply preparing cell extracts for use as a catalyst of cell-free protein synthesis by centrifugation, which improves cost effectiveness and productivity of cell-free protein synthesis. Specifically, a conventional process for preparing cell extracts comprises the complicated steps, i.e. cell culture, cell lysis, high-speed centrifugation, pre-incubation, dialysis and the like. In comparison, the cell lysate just obtained by centrifugation is directly applied to protein synthesis, thereby providing higher producibility and more consistent productivity of protein than the conventional process. Further, the cell extracts are prepared by the simple process to reduce the protein production cost and time by about 60% and about 80%, respectively.

### TECHNICAL FIELD

The present invention relates to cell extracts for cell-free protein synthesis and a process for cell-free protein synthesis using the same. More specifically, it relates to the cell extracts for cell-free protein synthesis, which are obtained by culturing cells in a culture medium, lysing the cultured cells and simply centrifuging the cell lysate, and contain cellular organelles and factors required for synthesis of a target protein, and to the process for cell-free protein synthesis using the cell extracts.

### BACKGROUND ART

Recently, sequences of genes in diverse organisms have been revealed with proceedings of various genome projects. As a result, it has been raised as facing problems to identify functions of numerous proteins encoded by the genes. According to a conventional recombinant gene technology, a protein is produced by a process comprising the multiple steps, i.e. gene cloning, introduction of the cloned gene to cells, culture of the cells with the introduced gene, lysis of the cultured cells, and isolation and purification of the protein from the cell lysate. However, this technology has marked limitations in terms of throughput to translate dramatically increasing novel genetic information to proteins.

Thus, cell-free protein synthesis is receiving renewed attention as an alternative to the conventional in vivo expression technology. According to the cell-free protein synthesis, intracellular machinery and factors related to protein synthesis are selectively extracted from cells, and synthetic processes of proteins are artificially repeated in an extracellular environment, out of control of physiological regulatory mechanism of cells, thereby producing a target protein on a large scale for a short period of time. Thus, a protein can be synthesized at a high rate without performing cell culture procedure. In addition, in the conventional in vivo expression technology, protein expression occurs in a defined space within the cell membrane or cell wall. By contrast, the cell-free protein synthesis uses the completely open system with no physical barrier, which gives an advantage to easily modify conditions for protein synthesis for various applications. For example, the cell-free protein synthesis is useful for protein production from genetic information within a few hours, as well as for selective labeling of protein molecules, ribosomal display, protein arraying on an immobilized surface and the like.

A system for cell-free protein synthesis had been initially used as a tool for addressing scientific questions in connection with translation of genetic information, and then, was first demonstrated by Zamecmick in 1958. Thereafter, various versions of cell-free protein synthesis systems have been developed heretofore. However, the prior cell-free protein synthesis systems have been limited in their wide uses and applications owing to their high cost for establishment.

The problem of high cost comes from a complicated and cost-consuming procedure for preparing cell extracts which serve as a catalyst for cell-free protein synthesis. For example, cell extracts derived from E. coli strains generally used for cell-free protein synthesis was prepared by the process proposed by Pratt in 1984, comprising the sequential steps of cell lysis, high-speed centrifugation (30,000 RCF), pre-incubation, dialysis and low-speed centrifugation (4,000 RCF). The preparation cost for the cell extracts accounts for about 30% or more of a total cost for cell-free protein synthesis. As described above, the preparation of the cell extracts has the problem of involving complicated and expensive steps. Therefore, it is crucial for solving the problem of high cost of cell-free protein synthesis to develop a more economic process for preparing cell extracts. In addition to the preparation cost, inconsistent protein productivity of the cell extracts due to the complicated preparation procedure has been an obstacle to commercialize a cell-free protein synthesis system. Nevertheless, researches for simplifying the procedures and improving the cost effectiveness of the preparation of cell extracts have not been intensively performed, while researches for improving productivity of proteins in cell-free protein synthesis have been vigorously performed.

### DISCLOSURE

### Technical Problem

An object of the present invention is to provide cell extracts used for cell-free protein synthesis, which are prepared by lysing cells cultured in a culture medium to give a cell lysate containing cellular organelles and factors required for synthesis of a target protein, and then, isolating the cell extracts by a simple centrifugation, to improve cost effectiveness and productivity.

Another object of the present invention is to provide a simple and economic process for cell-free protein synthesis in which the cell extracts obtained as above are applied to a cell-free protein synthesis system.

### Technical Solution

In order to achieve the above-described objects, the present invention provides cell extracts prepared by a process comprising the steps of:

lysing cells cultured in a culture medium to obtain a cell lysate containing cell organelles and factors required for synthesis of a target protein; and

centrifuging the cell lysate at 12,000˜30,000×g to obtain its supernatant.

In order to achieve another object, the present invention provides a process for cell-free protein synthesis, wherein the cell extracts are introduced to a reaction medium to obtain a target protein, the reaction medium comprising one or more L-amino acids selected from the group consisting of glycine (Gly, G), alanine (Ala, A), valine (Val, V), leucine (Leu, L), isoleucine (Ile, I), proline (Pro, P), phenylalanine (Phe, F), tyrosine (Tyr, Y), tryptophan (Trp, W), cysteine (Cys, C), methionine (Met, M), serine (Ser, S), threonine (Thr, T), lysine (Lys, K), arginine (Arg, R), histidine (His, H), aspartate (Asp, D), glutamate (Glu, E), asparagine (Asn, N) and glutamine (Gln, Q); an energy source for protein synthesis comprising one or more selected from the group consisting of ATP, CTP, GTP, TTP and UTP; genetic resources comprising DNA or mRNA which encodes the target protein; and a buffer solution.

### DESCRIPTION OF DRAWINGS

FIG. 1 is a flow chart schematically showing the processes for preparing the cell extracts (S12 Extract) according to the present invention and the conventional cell extracts (S30 Extract).

FIG. 2 is a graph showing the protein synthesis of the extract fractions obtained from each step of the conventional process for preparing the cell extracts (S30).

FIG. 3 is a graph showing the cell-free protein synthesis of the cell extracts of the invention (S12) and the conventional cell extracts (S30) depending upon the kinds of cells.

FIG. 4 is a graph comparatively showing the production cost and time of the cell extracts of the invention (S12) and the conventional cell extracts (S30).

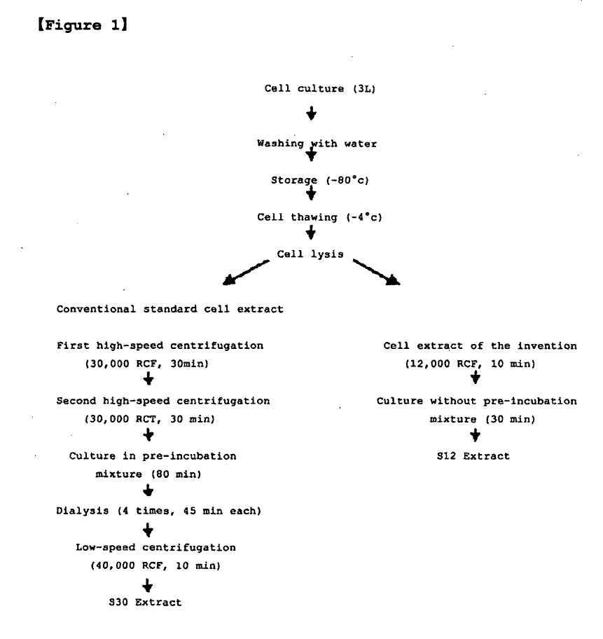

Other and further objects, features and advantages of the invention will appear more fully from the following description.

The technological or scientific terms used herein have their meanings usually understood by a person having ordinary skill in the art to which the present invention pertains, if not specifically defined otherwise. Explanations related to the same technical constitution and function as in conventional technologies are omitted herein.

According to the present invention, cell extracts used as a catalyst for cell-free protein synthesis are simply prepared by centrifugation, thereby improving cost effectiveness and productivity of cell-free protein synthesis. Specifically, while a conventional process for preparing cell extracts involves the complicated steps of cell culture, cell lysis, high-speed centrifugation, pre-incubation and dialysis, cell extracts are simply prepared by centrifugation, and used for protein expression directly, without involving any complicated steps, in the present invention. Thus, the cell extracts of the present invention show higher producibility and more consistent productivity of proteins than those prepared by the conventional process. Further, the cell extracts are prepared through the more simplified process, to reduce the production cost and time by about 60% and about 80%, respectively.

In the present invention, cultured cells are preferably prepared by culturing cells in a culture medium, centrifuging the cell culture to obtain cell pellets, rapidly freezing the cell pellets, and thawing the frozen cell pellets. The cells which have undergone freezing and thawing during the cell lysis readily release cellular organelles and factors required for protein synthesis from the cytoplasm to an extracellular medium.

The cells used in the present invention are preferably selected from the group consisting of E. coli, Bacillus subtilis, wheat germ, rice germ, barley germ, CHO cells, hybridoma cells and reticulocytes, but not limited thereto.

Amino acids and energy sources for protein synthesis according to the present invention are not limited to those components described above, but any amino acids and energy sources may be used if they can achieve the objects of the present invention.

A buffer solution should have suitable components and pH for the properties of a target protein, and so is not limited to specific ones having specific components.

Throughout the present specification, centrifugal force of centrifugation is expressed as relative centrifugal force (RCF) in the unit of ×g (gravity). In one embodiment, high-speed centrifugation is defined as centrifugation at 30,000×g, low-speed centrifugation is defined as centrifugation at 4,000×g, and simple centrifugation according to the present invention is defined as centrifugation at 12,000×g. Since the minimum centrifugal force for separating cellular organelles and factors related to protein synthesis from a cell lysate is 12,000×g, the cell lysate is centrifuged at 12,000˜30,000×g in the present invention. If the centrifugal force exceeds 30,000×g, extraction efficiency is not significantly increased while the production cost is remarkably increased.

In order to enhance protein synthesis, the cell extracts according to the present invention may further comprise conventional chaperone protein, a protease inhibitor, a nuclease inhibitor or a surfactant.

In the present invention, any processes for cell-free protein synthesis disclosed in the background may be used with the cell extracts of the present invention, and any processes, even though they have not been disclosed, may be used, as long as they conform to the objects of the present invention.

### BEST MODE

Hereinafter, the present invention will be described in more detail with reference to specific examples. However, the present invention is not limited by those examples, and it is apparent to a person having ordinary skill in the art that various alterations and modifications can be made within the spirit and scope of the invention.

### Example

### Preparation of Cell Extracts

Preparation of Cultured Cells

First, E. coli BL21 (DE3) [Novagen, Madison, U.S.A.] was cultured in a 3 L fermenter (2×YT medium) at 37° C. Then, in order to express T7 RNA polymerase for inducing transcription from a gene (DNA) encoding a target protein, when the absorbance (OD₆₀₀) reached 0.6, isopropylthio-β-D-galactoside (IPTG) was introduced to the fermenter at the final concentration of 1 mM. When the absorbance reached 4.5, cell culture was stopped, and cell pellets were selectively collected from the medium by centrifugation (4,500 RPM, 20 min, 4° C.)

Then, to the collected cell pellets was added 20 mL of buffer solution A [10 mM Tris-acetate buffer (pH 8.2), 14 mM magnesium acetate, 60 mM potassium glutamate, 1 mM dithiothreitol (DTT), 0.05% (v/v) 2-mercaptoethanol (2-ME)] per g of cells, and the mixture was thoroughly washed. The above centrifugation (4,500 RPM, 20 min) step was repeated three times. E. coli cells thoroughly washed as described above were stored in liquid nitrogen at −80° C.

(2) Preparation of a Cell Lysate

12.7 mL of Buffer solution B (wherein only 2-ME was excluded from buffer solution A) per 10 g of cells was added to the frozen E. coli cells obtained from Example (1), and the cells was homogeneously dispersed therein. By using a French press (Aminco), the cells were disrupted at a constant pressure (20,000 psi).

(3) Preparation of Cell Extracts

The cell lysate from Example (2) was simply centrifuged (12,000 RCF, 10 min, 4° C.) to obtain a supernatant, which was cultured (37° C., min) without pre-incubation to provide cell extracts. The obtained extracts were designated as S12 Extract (S12 Extract). The cell extracts according to the present invention (S12 Extract) were stored in liquid nitrogen before their use for cell-free protein synthesis.

### Comparative Example

From the cell lysate prepared according to the same procedure as in Examples (1) and (2), cell extracts were prepared according to the conventional process of Pratt.

First, the cell lysate was centrifuged at a high speed (30,000 RCF, 30 min, 4° C.) and the overlying lipid layer was removed therefrom to recover only the supernatant. The supernatant was centrifuged at the high speed (30,000 RCF, 30 min, 4° C.) once more again. Then, 3 mL of pre-incubation solution (293.3 mM Tris-acetate pH 8.2, 2 mM magnesium acetate, 10.4 mM ATP, 200 mM creatine phosphate, 4.4 mM DTT, 0.04 mM amino acids, 26.7 μg/mL creatine kinase) per 10 mL of supernatant was slowly added to the supernatant that had been centrifuged twice with thorough stirring, and pre-incubation was carried out in a darkroom at 37° C. for 80 minutes. The pre-incubated solution was introduced to a dialysis tube (10 kDa, SnakeSkin™ Pleated Dialysis Tubing, Rockford, U.S.A.), and dialyzed in 50-folds of buffer solution at 4° C. for 45 minutes four times to remove impurities of pre-incubation. The solution in the dialysis tube was centrifuged at a low speed (4,000 RCF, 10 min, 4° C.) to obtain cell extracts for protein synthesis. The extracts were designated as S30 Extract.

Then, S30 Extract was stored in liquid nitrogen before its use for cell-free protein synthesis.

### Experimental Example

In this Experimental Example, synthesis of the target protein from the cell extracts of the Example and the Comparative Example was measured, in order to evaluate the effects of the cell extracts of the present invention as compared to the conventional cell extracts.

(1) Cell-Free Protein Synthesis

Cell-free protein synthesis was carried out as follows.

First, in order to evaluate the ability of protein synthesis, gene (pK7-CAT) encoding chloramphenicol acetyltransferase (CAT) as the target protein was used.

For the cell-free protein synthesis, the cell extracts according to the present invention (S12 Extract) and those of Comparative Example (S30 Extract) were added to a standard reaction solution [57 mM Hepes-KOH (pH 8.2), 1.2 mM ATP, each 0.85 mM of CTP, GTP and UTP, 2 mM DTT, 0.17 mg/mL E. coli total tRNA mixture (from strain MRE600), 0.64 mM cAMP, 90 mM potassium glutamate, 80 mM ammonium acetate, 12 mM magnesium acetate, 34 μg/mL L-5-formyl-5,6,7,8-tetrahydrofolic acid (folinic acid), each 1.5 mM of 19 amino acids (except 0.5 mM of leucine), 2% PEG 8000, 67 mM creatine phosphate (CP), 3.2 μg/mL creatine kinase (CK), 0.01 M L-[U-¹⁴C] leucine (11.3 GBq/mmol, Amersham Biosciences), 6.7 μg/mL DNA (pK7-CAT)], respectively, to the concentration of 27% (v/v), and each mixture was homogeneously stirred and subjected to protein synthesis in an incubator (37° C.) for 3 hours.

A total amount of protein synthesized by cell-free protein synthesis was confirmed by measuring the amount of protein combined with ¹⁴C-leucine by means of radioisotope method (Kim and Swartz, 2000). The enzymatic activity of the synthesized protein (CAT) was confirmed by an optical method (Shaw, 1975).

(2) Evaluation of Ability of Protein Synthesis of the Cell Extract Fractions Obtained from Each Step of Preparing the Conventional Cell Extracts (S30 Extract)

First, cell extracts (S30 Extract) were prepared from E. coli BL21 (DE3) according to the conventional process disclosed by Pratt. The extract fractions obtained from each step for preparing S30 Extract were taken, and tested for their activity of protein synthesis.

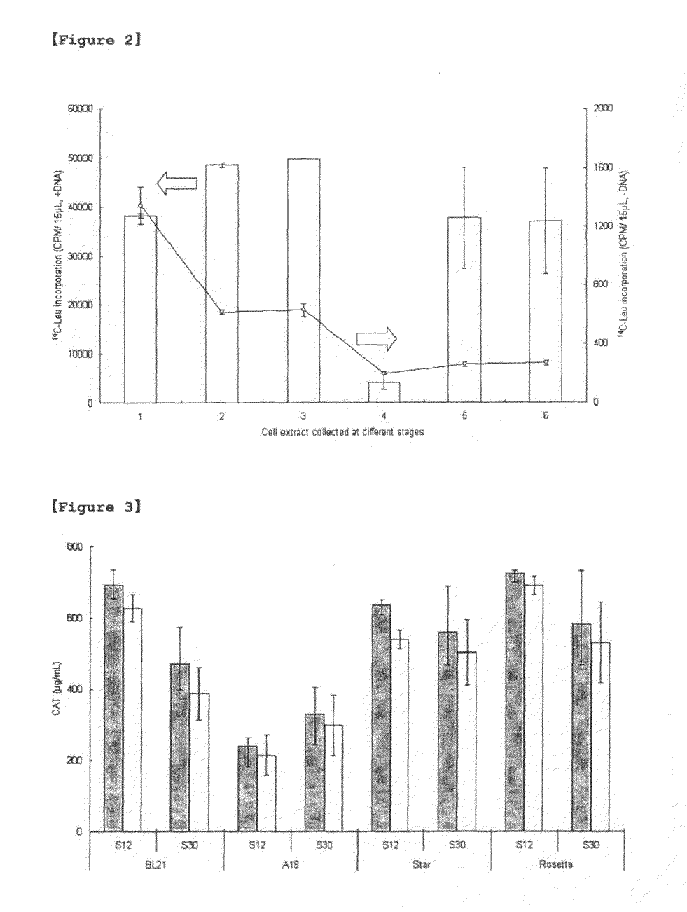

As a result, as can be seen from FIG. 2, it was surprisingly found that all cell extracts (including the finally obtained cell extracts) collected from each step of the preparation process of cell extracts showed similar ability of protein synthesis. Exceptionally, the cell extracts just after pre-incubation step showed noticeably low ability of protein synthesis. However, the cell extracts were confirmed to have the ability of protein synthesis recovered to the similar level to other samples after undergoing the dialysis step. In FIG. 2, 1 represents non-treated cell lysate, 2 represents the supernatant after undergoing the first centrifugation step, 3 represents the supernatant after undergoing the second centrifugation step, 4 represents the cell extracts pre-incubated with the pre-incubation solution, 5 represents the dialyzed cell extracts, and 6 represents the final cell extracts obtained by low-speed centrifugation of the dialyzed cell extracts.

From the above facts, it is presumed that low molecular weight substances (for example, inorganic phosphoric acid, etc.) accumulated through the pre-incubation step inhibit the protein synthesis, and the ability of protein synthesis is recovered by removing those substances through dialysis.

It is the most important in the present invention to find out that the crude cell lysate just after cell lysis in the conventional preparation process of cell extracts surprisingly shows the similar ability of protein production to the standard cell extracts (S30). Thus, it was concluded that pre-incubation and dialysis steps accounting for most of the production cost of cell extracts could be omitted, thereby to improve cost effectiveness.

(3) Cell-Free Protein Synthesis Using the Cell Lysate According to the Invention

As mentioned above, protein synthesis by using the cell lysate gives various advantages in terms of time and cost effectiveness. This Experimental Example was to confirm the properties of the cell lysate in protein synthesis, in order to apply it to cell-free protein synthesis.

The results are shown in Table 1. The measured values are mean values of the duplicated experiments.

First, the cell lysate had similar ability of protein synthesis to the standard cell extracts (S30 extract).

However, from the cell lysate, significant non-specific protein expression (background expression) was observed. That is, non-specific protein expression was significantly higher than 0.9% (the value observed in the standard cell extracts (S30 Extract)), and constitutes 3.5% of a total production amount of the target protein, as measured by radioisotope method. This means that protein synthesis occurs from genetic materials (mRNA, DNA etc.) in the cell lysate, so that the cell lysate is not appropriate for direct application to cell-free protein synthesis. Further, the cell lysate is difficult to handle with a micropipette, owing to its high viscosity.

However, as can be seen from Table 1, such problems can be substantially solved by simply centrifuging the cell lysate (12,000 RCF, 10 min, 4° C.) to obtain the cell extracts according to the present invention (S12 Extract). The problems of too high viscosity and poor handability were completely solved, and non-specific expression was reduced by about 34% through simple centrifugation. In particular, the supernatant of the cell lysate after simple centrifugation showed enhanced ability of protein synthesis by 28%. In case of expressing the gene (pK7-CAT) from the cell extracts, the productivity was increased by 1.5-folds compared to expression of the gene from the standard cell extracts (S30 Extract).

In order to further solve the problem of non-specific expression, the supernatant of cell lysate was cultured in an incubator without the pre-incubation solution for various periods of time. As a result, the supernatant of cell lysate cultured for about 30 minutes had not only non-specific expression reduced to 1% or less but also slightly enhanced ability of protein synthesis.

TABLE 1

| | | | | Non-specific |
|---|---|---|---|---|
| | Culture | ¹⁴C-leucine bond (CPM/15 μl) | | protein |
| Cell extract | time | (−DNA) | (+DNA) | expression(%) |
| Crude extract | — | 1343 ± 128 | 38068 ± 462 | 3.5 |
| S30 Extract | — | 316 ± 64 | 34533 ± 9035 | 0.9 |
| S12 Extract | 0 | 844 ± 18 | 48763 ± 676 | 2.3 |
| | 10 | 729 ± 42 | 49921 ± 1280 | 1.5 |
| | 20 | 552 ± 49 | 53428 ± 903 | 1.0 |
| | 30 | 506 ± 51 | 53965 ± 1228 | 0.9 |
| | 40 | 447 ± 21 | 52732 ± 201 | 0.8 |
| | 50 | 456 ± 26 | 51848 ± 1150 | 0.9 |
| | 60 | 393 ± 21 | 50027 ± 1054 | 0.8 |
| | 70 | 346 ± 13 | 51372 ± 747 | 0.7 |
| | 80 | 375 ± 11 | 51091 ± 427 | 0.7 |
| | 90 | 304 ± 32 | 49440 ± 398 | 0.6 |

(4) Cell-Free Protein Synthesis Depending Upon the Kinds of Cells

In order to confirm whether the simplified preparation process of cell extracts according to the present invention is applicable to other kinds of E. coli cells, cell extracts were prepared according to the processes of the Example and the Comparative Example, by using 4 kinds of known cells (Rosetta (DE3), BL21 (DE3), BL21-star (DE3), A19 (DE3)) widely used as materials for preparation of cell extracts for cell-free protein synthesis. Cell-free protein synthesis was performed according to the same procedure as in Experimental Example (1). Strain A19 is derived from E. coli strain K12, and other three strains are derived from E. coli strain B.

As a result, as shown in FIG. 3, the cell extracts of the invention (S12 Extract) showed higher productivity of the target protein (CAT) than the conventional cell extracts (S30 Extract), in most cases. Further, more consistent productivity was obtained by using the cell extracts of the present invention. Only, in case of strain A19 derived from E. coli K12, the conventional cell extracts had slightly higher protein production than the cell extracts of the present invention. In FIG. 3, filled bars show the total amount of the synthesized CAT, and open bars show enzymatic activity of CAT.

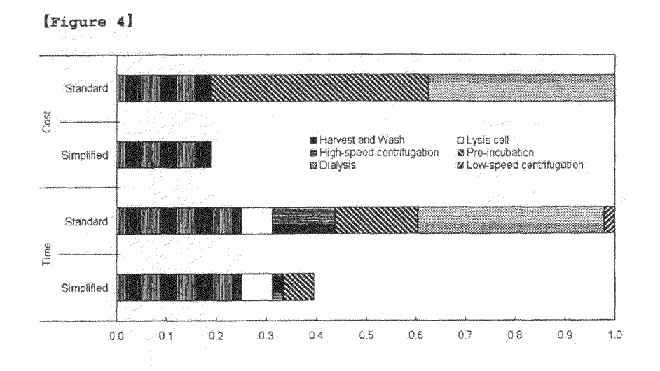

Thus, it was found that the cell extracts (S12 Extract) prepared according to the present invention had excellent time and cost effectiveness, and high rate of specific protein synthesis as compared to the cell extracts (S30 Extract) prepared according to the conventional process. FIG. 4 shows the relative production cost and time of the cell extracts of the present invention to the production cost (72 USD) and time (8 hr) of the conventional cell extracts. As a result, the cell extracts according to the present invention can carry out cell-free protein synthesis at a cost of 20% for a period of time of 40% as compared to the cell extracts (S30 Extract) prepared according to the conventional process. Moreover, the cell extracts according to the present invention had the increased ability of protein expression by 1.5-folds.

### INDUSTRIAL APPLICABILITY

As described above, the cell extracts according to the present invention can be obtained by a more simplified process thereby to reduce the production cost and time by about 60% and about 80%, respectively, and further, shows higher ability of protein production and more consistent productivity than the conventional cell extracts.

Modifications, alterations and replacements in a certain range can be made to the disclosure described above, and only a part of the characteristics of the present invention may be used. Thus, the attached claims should be interpreted broadly and to conform the spirit and scope of the present invention.

1. A process for cell-free protein synthesis by using cell extracts, which comprises the steps of:
lysing cells cultured in a culture medium to obtain a cell lysate containing cellular organelles and factors required for synthesis of a target protein;
centrifuging said cell lysate at 12,000˜30,000×g to obtain its supernatant to prepare cell extracts; and
introducing said cell extracts to a reaction medium containing one or more amino acids, an energy source for protein synthesis, genetic resources and a buffer solution.

2. The process for cell-free protein synthesis according to claim 1, wherein said cells are selected from the group consisting of E. coli, Bacillus subtilis, wheat germ, rice germ, barley germ, CHO cells, hybridoma cells and reticulocytes.

3. The process for cell-free protein synthesis according to claim 1, wherein said cell lysate is centrifuged at 12,000×g.

4. The process for cell-free protein synthesis according to claim 1, wherein the amino acid(s) comprise(s) one or more L-amino acids selected from the group consisting of glycine, alanine, valine, leucine, isoleucine, proline, phenylalanine, tyrosine, tryptophan, cysteine, methionine, serine, threonine, lysine, arginine, histidine, aspartate, glutamate, asparagine and glutamine; the energy source for protein synthesis is one or more selected from the group consisting of ATP, CTP, GTP, TTP and UTP; and, the genetic resources comprise DNA or mRNA encoding the target protein.
